(19) United States
(12) Patent Application Publication
(10) Pub. No.: US 2009/0177187 A1
(43) Pub. Date: Jul. 9, 2009

(54) Pressure Activated Valve with Angled Slit

(76) Inventors: Karla Weaver Quigley, Framingham, MA (US); Steven Grantz, Pelham, NH (US); Richard Pok, Malden, MA (US); Anthony Hien, Stoneham, MA (US); Kimberly Un, Lawrence, MA (US)

Correspondence Address: FAY KAPLUN & MARCIN, LLP, 150 BROADWAY, SUITE 702, NEW YORK, NY, 10038, US

(21) Appl. No.: 12/207,874

(22) Filed: Sep. 10, 2008

Related U.S. Application Data
(60) Provisional application No. 60/971,356, filed on Sep. 11, 2007.

(51) Int. Cl. A61M 25/14; F16K 15/14

(52) U.S. Cl. 604/537; 137/845; 604/247

(57) ABSTRACT

A pressure actuated valve comprises a first membrane having a slit extending through the membrane at a nonzero draft angle relative to a perpendicular to a surface of the membrane, material of the membrane biasing the slit closed so that the slit remains closed when a fluid pressure applied to the membrane is below a threshold level and, when the fluid pressure is at least the threshold level, edges of the slit separate from one another to permit fluid flow through the membrane.

### PRIORITY CLAIM

The present application claims priority to U.S. Provisional Patent Application Ser. No. 60/971,356 filed on Sep. 11, 2007 entitled “Pressure Activated Valve With Angled Slit.” The entire disclosure of this application is expressly incorporated herein by reference.

### BACKGROUND OF THE INVENTION

Medical procedures often require repeated and prolonged access to the vascular system. For example, a dialysis catheter may be implanted to form a semi-permanent conduit to and from a blood vessel for the removal and/or introduction of blood, fluids, medications, chemotherapy agents, nutrients, etc. The catheter must be sealed from the outside environment when not in use to prevent the leakage of fluids therefrom and to prevent external contaminants and air from entering the body.

These catheters are often sealed between therapeutic sessions by applying clamps thereto. However, the repeated application of such clamps may weaken catheter walls as stress is repeatedly applied to the same locations on the catheter walls. In addition, this clamping may result in an imperfect seal allowing air or other contaminants to enter the catheter entailing a risk of infection and/or coagulation of the blood.

### SUMMARY OF THE INVENTION

In one aspect, the present invention is directed to a pressure actuated valve comprising a first membrane having a slit extending through the membrane at a nonzero draft angle relative to a perpendicular to a surface of the membrane, material of the membrane biasing the slit closed so that the slit remains closed when a fluid pressure applied to the membrane is below a threshold level and, when the fluid pressure is at least the threshold level, edges of the slit separate from one another to permit fluid flow through the membrane.

### BRIEF DESCRIPTION OF THE DRAWINGS

FIG. 1 shows a perspective view of a catheter housing with a slitted membrane valve according to an embodiment of the invention;

FIG. 2 shows a schematic side elevation view of a slitted membrane with a slit cut at an angle according to an embodiment of the invention;

FIG. 3 shows a front view of a slitted membrane according to an embodiment of the invention;

FIG. 4 shows a perspective view of the slitted membrane shown in FIG. 3;

FIG. 5 shows a front view of a slitted membrane with 0 deg. Draft angle;

FIG. 6 shows a perspective view of the slitted membrane shown in FIG. 5;

FIG. 7 is a chart showing bench test results for several slitted membranes according to the invention;

FIG. 8 is a second chart showing results for several slitted membranes according to the invention;

FIG. 9 is a graph showing results of a cut angle study for several slitted membranes according to the invention;

FIG. 10 is a diagram of a slitted membrane according to an embodiment of the present invention having two parallel slits;

FIG. 11 is a diagram of a slitted membrane according to another embodiment of the invention having symmetrical slits; and

FIG. 12 shows a perspective view of a catheter housing with a membrane cartridge for a valve according to a further embodiment of the invention.

### DETAILED DESCRIPTION

The present invention may be further understood with reference to the following description and the appended drawings, wherein like elements are referred to with the same reference numerals. The present invention is related to devices for accessing the vascular system and, in particular, to pressure activated valves sealing catheters facilitating chronic access to a blood vessel. Typical pressure activated valves comprises two principal components: 1) a valve housing an end of which is coupled to the catheter while the other end is coupled to an external device; and 2) a slitted membrane sandwiched between male and female halves of the housing.

Pressure activated valves automatically seal catheters as they are biased closed (e.g., by elastic properties of the material of the membrane) so that edges of the slit are moved apart from one another to permit fluid flow therethrough only when a fluid pressure applied thereto exceeds a predetermined threshold level. For example, the threshold level may be chosen to be above a level of pressure to which the valve is expected to be subjected through natural operation of the vascular system (e.g., fluctuations in venous pressure) and below a level of pressure which will be applied by a device (e.g., a dialysis machine) to be coupled to the catheter when it is in use. When no fluid is present or when the fluid pressure applied thereto is below the threshold value, the slit remains closed and no fluid passes through the valve. The slits of many conventional pressure activated valves extend through the membrane substantially perpendicular to the surface of the membrane. However, under certain conditions, such designs may not prevent bleed back (I.e., the flow of blood through the catheter out of the patient).

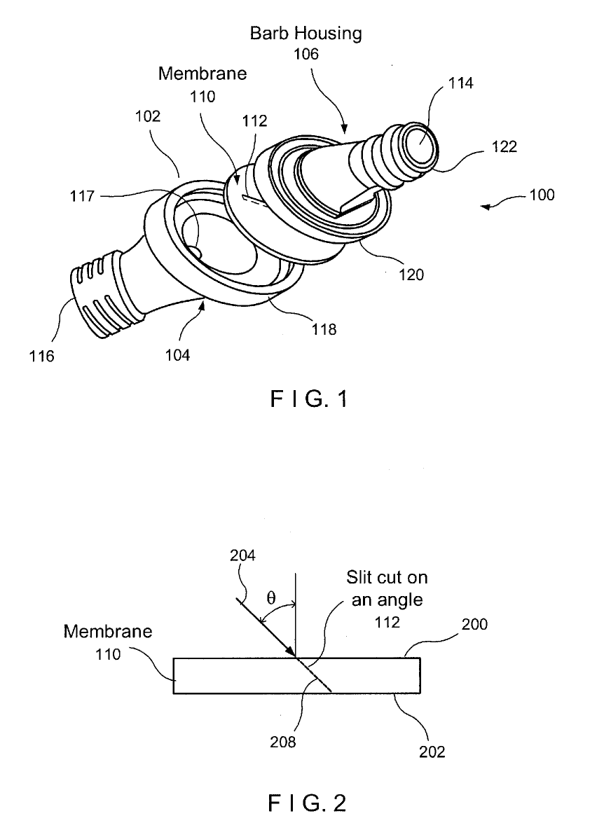

As shown in FIG. 1, a valve 100 according to an exemplary embodiment of the invention a slitted membrane comprises a housing 102 divided in two halves, a luer housing 104 and a barb housing 106. The luer housing 104 connects to a catheter (not shown) via a connector 116, so that the flow passage 112 forms a continuous flow path with the working channel of the catheter. The luer housing 104 has a female portion 118 that mates with the male portion 120 of the barb housing 106 to fluidly connect the flow passage 112 to the flow passage 114. The barb housing 106 comprises a barb 122 for fluid connection with external tubing.

A membrane 110 sandwiched between the barb housing 106 and the luer housing 104 comprises a slit 112 that extends through the thickness of the membrane 110. When a sufficient fluid pressure is applied to the membrane 110 (i.e., a pressure above a threshold pressure of the valve 100), the slit 112 opens against a closing force exerted due to resilience of the material of the membrane 110 and the geometry of the slit. When a pressure below a threshold value is applied, the closing force maintains the slit closed preventing fluid from passing through the valve 100. As described above, the membrane 110 may, for example, be designed so that the threshold is greater than the amplitude of normal pressure fluctuations of the vascular system and lower than a pressure to which the membrane 110 will be subject when the valve 100 is in use (e.g., when hooked to an external device to receive blood from or transfer blood or other products to a blood vessel).

According to the embodiments of the invention, the slit 112 is cut through the membrane 110 at an angle relative to an axis perpendicular to a surface of the membrane 110. As shown in FIG. 2, the membrane 110 has surfaces 200, 202 oriented substantially perpendicular to a direction of flow through the lumen between the barb housing 106 and the luer housing 104. In the exemplary embodiment, the slit 112 is cut at a draft angle θ, along a line 204 relative to the axis perpendicular to the surface 200 of the membrane 110. In contrast to conventional slits that are cut perpendicular to the surface of the membrane 110, with a draft angle θ=0 degrees, the angle at which the slit 112 is cut increases a surface area 208 of the portions membrane 110 facing each other across the slit 112. This increases the sealing footprint of the membrane 110, consequently increasing the closing force exerted on the slit 112. A range of desired angles θ will vary as a function of valve membrane compression, slit length, thickness & durometer of the material. In a preferred embodiment, the range will be between 0.1 and 0.9 degrees for a slit 0.360″ long in a membrane having a thickness of between 0.0160″ and 0.0165″ formed of a material having a durometer of between 63 A and 65 A and a compression of approximately 500 grams.

Increasing the draft angle θ of the slit 112 is beneficial to a point. If the draft angle θ becomes too large, the ability of the membrane 110 to reseal when not in use decreases as the tension on the membrane 110 biasing the slit 112 toward the closed position decreases as this angle is increased beyond a threshold level. An optimal draft angle θ thus can be derived for slitted membranes of various properties. Thus an optimal draft angle θ varies as a function of several variables including, among others, the desired threshold pressure, the material of which the membrane 110 is formed, dimensions of the membrane 110 and the length of the slit 112 along the surfaces 200, 202 of the membrane 110.

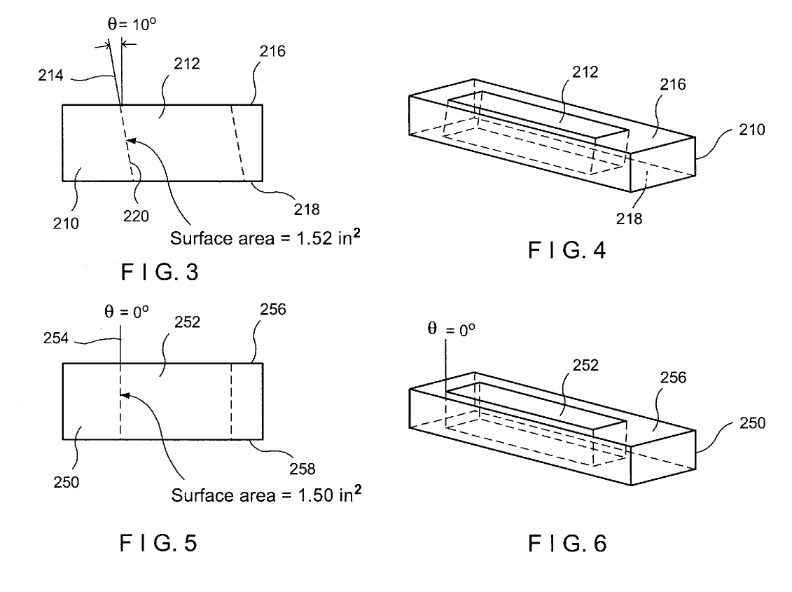

FIGS. 3 and 4 show, respectively, a front view and a perspective view of an exemplary embodiment of a slitted membrane 210 according to the invention, which forms a sealing element of a pressure activated valve. For example, the membrane 210 may be formed of silicone with a slit 212 extending from a first surface 216 to a second surface 218. According to the invention, the exemplary slit 212 is cut at a draft angle θ=10 degrees, along a line 214 inclined from an axis perpendicular to the surface 216. Those skilled in the art will understand that the surface area 220 will be equal to the surface area of a plane extending through the membrane 210 on a perpendicular divided by cosine 2. If the exemplary membrane 210 is 0.5″ thick and the length of the slit 212 along the surface 216 is 3.0″, a surface area 220 of the slit 212 is equal to 1.52 in² for 2=10E (as opposed to 1.5 in² for a conventional slitted membrane in which θ=0 degrees, as shown in FIGS. 5 and 6).

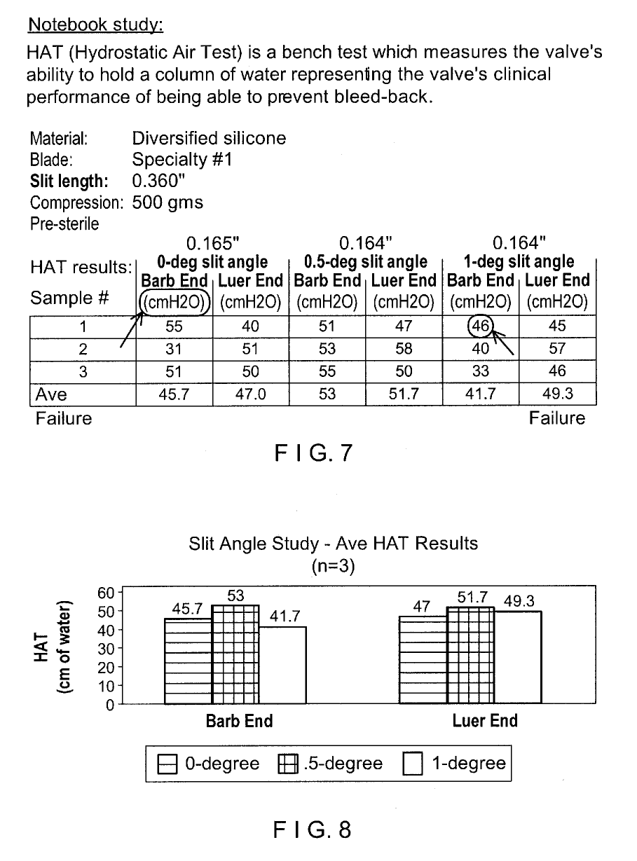

A hydrostatic air test (HAT) was carried out to determine the optimal non-zero draft angle for exemplary slitted membranes having set dimensions. As indicated above, although cutting the slit at an angle increases the sealing surface area and reduces bleed-back, an excessive angle decreases the ability of the membrane to reseal. The HAT is a bench test that measures the valve's ability to hold a column of water, and thus is representative of the valve's ability in a clinical setting to prevent bleed-back. FIG. 7 shows results for the testing of an exemplary valve comprising a diversified silicone slitted membrane with a slit length of 0.360″ and a compression of 500 grams. Those skilled in the art will understand that the term compression as used herein refers to an amount of force applied to the membrane when joining the two housings together. If compressive force/sealing pressure is too great then the membrane moves toward the lowest pressure area (slit) & creates a pucker, reducing the ability of the membrane to seal. As would be understood by those skilled in the art, this may lead to problems such as bleedback, reflux, air embolism, etc. When compression is less than desired, blood may leak around the membrane. The desired range of compression is a function of slit length, thickness & durometer of the material (e.g., silicone). In a preferred embodiment, the desired compression range is from 500 to 750 grams.

The results shown in FIG. 7 comprise three sample membranes for each tested draft angle. The resistance of the membranes to hydrostatic pressure was measured for draft angles of 0.0 degrees, 0.5 degrees and 1.0 degree. Measurements were taken at the barb end and at the luer end of the valve. As shown, the average failure pressures at the barb end and luer end for the 0.0 degree slit are 45.7 cm H₂O and 47.0 cm H₂O. For the 0.5 degree slit the corresponding values are 53.0 cm H₂O and 51.7 cm H₂O. For the 1.0 degree slit the values are 41.7 cm H₂O and 49.3 cm H₂O. These averaged results are shown in bar graph format in FIG. 8.

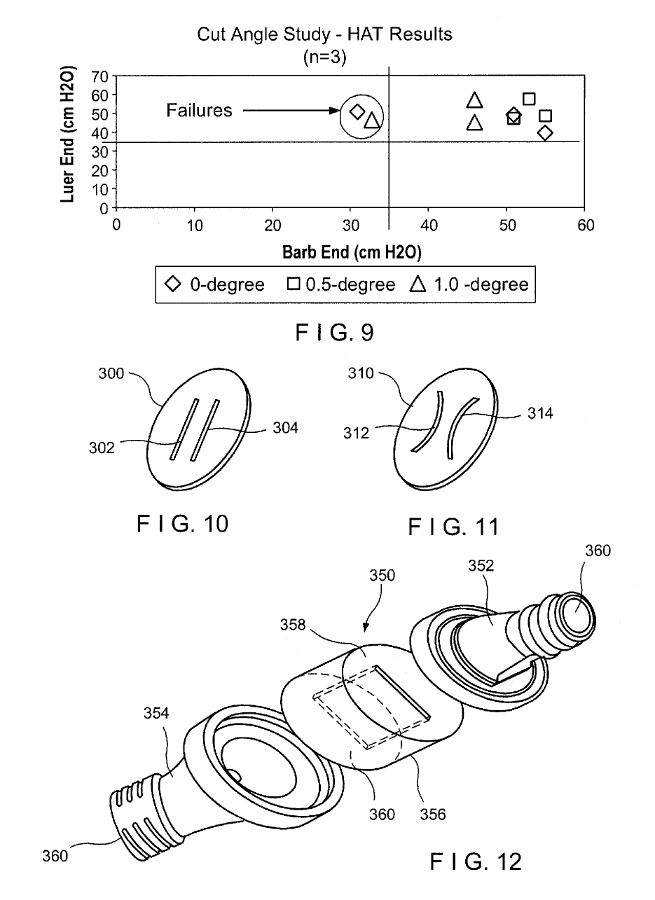

A different representation of the test results is shown in FIG. 9. The HAT results for different slit cut angles are shown by plotting the pressure withstood by the membrane as measured at the luer end versus the pressure at the barb end. The membrane failures are also shown in the plot, identifying the membranes that did not withstand the minimum acceptable pressure of 35 cm H₂O.

As can be seen from the diagrams and plots, for the exemplary membrane tested, a slit cut at a draft angle of approximately 0.5 degrees yielded the best HAT results. That is, for the exemplary membrane made of diversified silicone having the specified dimensions, the greatest resistance to bleed-back can be expected when the slit is cut with a draft angle of approximately 0.5 degrees. The exemplary embodiment of the invention thus reduces the risk of bleed-back while maintaining the desired threshold pressure for a specified thickness.

As shown in FIG. 10, a membrane 300 according to a further embodiment of the invention includes parallel slits two parallel slits 302, 304. Alternatively, the slits may be symmetrical with respect to the X or the Y axis of the membrane, or may be symmetrical with respect to a center of the membrane. For example, the membrane 310 shown in FIG. 11 comprises slits 312, 314 that are symmetrical with respect to the axes of the membrane 310.

In yet another embodiment, the slitted membranes according to the invention may be pre-mounted in a cartridge or holding fixture which is incorporated within the housing. For example, as shown in FIG. 12, a valve housing 350 may comprise a barb housing 352 and a luer housing 354 that fit together to form a flow passage 360. A membrane cartridge 356 is incorporated between the barb housing 352 and the luer housing 354, within the flow passage 360. As shown, the membrane cartridge 356 comprises two slitted membranes 358, 360. However, as would be understood by those skilled in the art, a single slitted membrane or additional slitted membranes may be disposed therein. Multiple membranes may also be sandwiched between portions of the housing, without a cartridge to hold them.

The present invention has been described with reference to specific embodiments, and more specifically to a venous dialysis catheter. However, other embodiments may be devised that are applicable to other medical devices, such as any catheter sealed using pressure activated valve technology, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A pressure actuated valve, comprising a first membrane having a first slit formed therethrough, material of the first membrane biasing the first slit closed so that the first slit remains closed when a fluid pressure applied to the first membrane is below a first threshold level and, when the fluid pressure is at least the first threshold level, edges of the first slit separate from one another to permit fluid flow through the first membrane, the first slit extending through the first membrane at a first nonzero draft angle relative to a perpendicular to a surface of the first membrane.

2. The pressure actuated valve according to claim 1, further comprising a housing including a cartridge received therein, the membrane being mounted within the cartridge.

3. The pressure actuated valve according to claim 2, further comprising a second membrane mounted within the cartridge, the second membrane having a second membrane slit formed therethrough, material of the second membrane biasing the second membrane slit closed so that the second membrane slit remains closed when a fluid pressure applied to the second membrane is below a second threshold level and, when the fluid pressure is at least the second threshold level, edges of the second membrane slit separate from one another to permit fluid flow through the second membrane, the second membrane slit extending through the second membrane at a second nonzero draft angle relative to a perpendicular to a surface of the second membrane.

4. The pressure actuated valve according to claim 1, wherein the first membrane includes a second slit extending therethrough.

5. The pressure actuated valve according to claim 4, wherein the first and second slits are symmetric with respect to one of a line extending in a plane of the surface of the first membrane and a center of the first membrane.

6. The pressure actuated valve according to claim 1, wherein the first draft angle is less than about 10 degrees.

7. The pressure actuated valve according to claim 1, wherein the first draft angle is about 0.5 degrees.

8. The pressure actuated valve according to claim 1, wherein the first draft angle is selected to maximize a closing force of the first slit.

9. The pressure actuated valve according to claim 1, wherein the first draft angle is selected to reduce bleed-back of the pressure actuated valve.

10. A catheter, comprising:
a valve housing attached with a fluid passage extending threrethrough; and
a first slitted membrane mounted across the passage of the valve housing, the first slitted membrane defining a first slit cut at a first non-zero draft angle relative to a first axis substantially perpendicular to a surface of the first membrane.

11. The catheter according to claim 10, wherein the first slitted membrane is formed of silicone.

12. The catheter according to claim 10, wherein the first draft angle is approximately 0.5 degrees.

13. The catheter according to claim 10, wherein the first draft angle is less than approximately 10 degrees.

14. The catheter according to claim 10, wherein the first draft angle is selected to obtain a desired sealing footprint of the first slit.

15. The catheter according to claim 10, wherein the first membrane comprises a second slit cut at a non-zero draft angle.

16. The catheter according to claim 10, further comprising a second slitted membrane mounted across the passage of the valve housing the second slitted membrane defining a second membrane slit cut at a second draft angle from a second axis substantially perpendicular to a surface of the second membrane.

17. The catheter according to claim 16, further comprising a cartridge holding the first and second membranes.
